(19) United States
(12) Patent Application Publication
(10) Pub. No.: US 2020/0005938 A1
(43) Pub. Date: Jan. 2, 2020

(54) REMOTE PATIENT MONITORING SYSTEM

(71) Applicant: Wachter, Inc., Lenexa, KS (US)

(72) Inventors: Brian Sloan, Lenexa, KS (US); Sean Finnegan, Mt. Laurel, NJ (US); Phil Kennedy, Lenexa, KS (US)

(21) Appl. No.: 16/442,433

(22) Filed: Jun. 14, 2019

Related U.S. Application Data
(60) Provisional application No. 62/685,270, filed on Jun. 14, 2018.

(51) Int. Cl. G16H 40/67; G16H 40/40

(52) U.S. Cl. CPC G16H 40/67; G16H 40/40

(57) ABSTRACT

A patient monitoring system for audiovisual observation and communication between a remote patient observation station and a patient hospital room to provide continuous monitoring of patient condition and status. Embodiments of the system include a management server to provide centralized operation and administration of one or more media servers. Each media server provides centralized operation and administration of one or more monitoring devices, which may include platforms such as mobile wheeled carts, mobile wall units, and fixed cameras and intercoms.

### CROSS REFERENCE TO RELATED APPLICATION

This application claims the benefit of the prior-filed, co-pending provisional patent application, Ser. No. 62/685,270, filed Jun. 14, 2018.

### FIELD OF THE INVENTION

The present invention relates to a system and method for providing audio and video monitoring and communications between a hospital patient and remotely located hospital personnel.

### BACKGROUND OF THE INVENTION

Most hospitals, ICU units, or similar medical facilities, comprise patient rooms proximate to and often surrounding a nurse's station where nursing staff can obtain patient information electronically from electronic patient medical records and quickly move from the centralized station to patient rooms as needed. While most patients do not require constant supervision, but only period checks by nurses and other hospital staff, some patients do require constant or near constant monitoring.

Presently in the US, there are approximately 3 million nurses serving an overall population of approximately 320 million. It has been estimated that, due to an aging population, the US will need 4 million nurses by the year 2020 to adequately care for patients. Based on projected graduation rates from nursing schools, the actual number of new nurses will fall short of this target.

There exist alternate potential means of monitoring a patient, such as video cameras linked to a video screen observable at a central station, similar to a typical security camera system. It can be costly and disadvantageous, however, to place security cameras in each patient room especially since, at any given time, most of the cameras will not be needed and also since each camera represents a potential for violation of patient privacy if not used and monitored correctly. In addition, while cameras can be useful to monitor a patient they do not provide the interaction often required when monitoring a seriously ill patient, such as the ability to interact verbally with the patient.

Therefore, there exists a need for a system and apparatus for monitoring patients in an interactive way that accounts for patient privacy needs and can more readily substitute for in person monitoring.

### SUMMARY OF THE INVENTION

Embodiments of the present invention may comprise a video camera for monitoring a patient in a hospital bed within a patient room. An intercom, such as an Internet Protocol (IP) and Session Initiation Protocol (SIP) intercom, is also provided for communicating verbally with the patient and for monitoring room audio. In order to view the patient at night, an infrared (IR) lamp/illuminator may be provided to illuminate the room in a manner that will not disrupt the patient's sleep. In such cases, a camera capable of taking or providing images under illumination by infrared radiation is provided.

In some situations, patients and nurses may not speak the same language. Therefore, even if hospital personnel are available for person to person monitoring, communication may still be a difficulty. In certain embodiments of the present invention, the audio or intercom system will play and visually display preset or prerecorded messages in the patient's language.

In certain embodiments of the invention, the camera, IR lamp, microphone and speaker of audio system are housed in a wall mounted unit. The wall mounted unit may be powered either by a proximate wall outlet or hard wired to an electrical line running within the wall. Communication with system servers and the operator station may be via Ethernet or other hard wire cabling or via WIFI or other radio signal. In other embodiments of the invention, the camera, IR lamp, and audio system components may be provided on a mobile cart. In preferred embodiments, the cart comprises a wheeled base for rolling the cart from patient room to patient room as-needed. An elongated tower arm extends generally vertically upward from the base and engages telescopically with a second elongated boom which extends generally vertically to a desired height, typically a few inches below the ceiling level. This vertically extending or telescoping boom may then be lowered in order to pass through door frames when moving the cart in or out of a patient room, and further allows the tower to be extended upward when in a patient room to maximize and optimize tower height. A locking mechanism may be used to fix the boom at a selected height. In some embodiments, either or both of the tower arm and boom comprise extruded aluminum.

Since the camera may be mounted near the top of the upwardly extending boom, maximizing the boom height allows the camera to be placed also at a maximized/optimized height so that the lens of the camera may be directed or angled outward and downward to capture images of a patient, who is typically reclined in a substantially horizontal disposition in a hospital bed. Typically proximate to the camera, an IR lamp is mounted on the boom and is pointed in generally the same direction as the lens of the camera to illuminate the area of the room to be imaged by the camera.

An upper portion of the tower may include a bracket holding a small, planar, horizontally-disposed table, shelf or tray. The tray can provide means for mounting a computer processor, monitor and keyboard. (Alternatively, a touch screen monitor may be directly connected, via hardware or radio signal, to a system server.) The computer processor may receive and control signals emanating from the camera and audio system, as well as providing means to control illumination of the IR lamp. The processor may be connected to the rest of the system and, therefore, to the operator station, via Ethernet or other signal conducting cabling, or via WIFI or other radio signal.

The base of the cart is typically provided with casters or other types of wheels or rolling elements so that the cart may be rolled across a floor surface. It should be appreciated, however, that the cart base may simply comprise legs or a flat, base structure, that can rest upon a separate, prior art dolly or other prior art wheeled cart for transporting the cart between rooms.

An LED or other light source is provided to illuminate when the video signal from the cart camera is disengaged so that personnel within the room will be assured that video images are no longer being transmitted from the cart and outside of the room, and that they may proceed with patient care with patient privacy assured.

At a Patient Observation Station according to the present invention, a video monitor is provided for the operator to view one or more patient rooms having observation carts or wall units according to the present invention. Video feeds from these patient rooms may each be presented on their own corresponding monitor at the Patient Observation Station, or multiple video feeds from multiple observation carts or wall units may be displayed in frames or tiles upon a single video monitor screen. The video monitor screen receives video from either a local video processor (such as a local CPU) at the Patient Observation Station or from a centralized server. Similarly, a Patient Observation Station will include a speaker, or other means of listening to audio, such as headphones, and a microphone. These audio components are connected to the local processor, or more remotely to a system server.

Embodiments of a Remote Patient Monitoring System include a Management Server as the primary administrative and user services platform and one or more managed and associated Media Servers the function as asset (e.g. Monitoring Device) managers. Monitoring Devices may include Mobile Cart assets, Portable Wall-mounted assets and Fixed Camera assets.

Embodiments of a patient monitoring system may comprise a management server, a media server, and a monitoring device. The monitoring device may include a computer processor, a video camera, a microphone, and wireless router for transmitting video, audio and data signals to the media server and management server. In some embodiments of the system, video signals from the video camera are conveyed via the media server to a patient observation station for viewing by a patient observer, and audio signals from the video camera are conveyed via the media server to a patient observation station for viewing by a patient observer. In some embodiments of the system, a patient observer may select a privacy mode to selectively darken a portion of a patient observation station monitor screen in order to obscure video images from a patient room. In such case, selecting a privacy mode causes a privacy indicator light to dim in a patient room, thereby confirming to the room occupants that video signals taken by the camera are no longer being displayed upon the monitor screen.

Other advantages of the invention will become apparent from the following description taken in connection with the accompanying drawings, wherein is set forth by way of illustration and example several embodiments of the present invention.

### BRIEF DESCRIPTION OF THE DRAWINGS

FIG. 1 is a diagram illustrating components of an embodiment of the system of the present invention.

FIG. 2 is a diagram further illustrating components of an embodiment of the system of the present invention.

FIG. 3 is a perspective view of an exemplary patient observation station and workstation.

FIG. 4 is a diagram illustrating connectivity between management and media servers, workstations, monitoring devices and other system assets.

FIG. 5 is a diagram illustrating a management server console.

FIG. 6 is diagram illustrating selected components of a first mobile cart platform.

FIG. 7 is a perspective view of an embodiment of first mobile cart.

FIG. 8 is a front elevation of the first mobile cart of FIG. 7.

FIG. 9a shows a side elevational view of a first mobile cart with the boom extended fully upward as deployed for use in a patient room.

FIG. 9b shows a side elevational view of a first mobile cart with the boom retracted for movement or storage of the cart.

FIG. 10 is a side elevation of the first mobile cart showing movement of the keyboard tray between retracted and extended positions.

FIG. 11 is a perspective view of an alternative embodiment of a mobile cart.

FIG. 12 is a front perspective view of a portable wall asset.

FIG. 13 is a front view of a portable wall asset.

FIG. 14a is a front perspective view of a female bracket assembly for mounting on a wall and on which to removably hang a portable wall asset.

FIG. 14b is a rear perspective view of a portable wall asset showing male brackets attached to the rear surface of the portable wall asset.

FIG. 15 is a front perspective view of an alternative embodiment of a portable wall asset.

FIG. 16a is a front elevation of the portable wall asset of FIG. 15.

FIG. 16b is a side elevation of the portable wall asset of FIG. 16a.

FIG. 17a is a front perspective view of a female bracket assembly for mounting on a wall and on which to removably hang a portable wall asset.

FIG. 17b is a rear perspective view of the portable wall asset of FIG. 15 showing male brackets attached to the rear surface of the portable wall asset.

FIG. 18 is a diagram showing a stand-alone intercom and fixed-lens camera connected to the System via an Ethernet switch.

FIG. 19 is a diagram of a System dashboard presented via a graphic user interface on a workstation computer monitor.

FIG. 20 is a diagram illustrating an alternative embodiment of a System.

FIG. 21 is a diagram illustrating a communication control screen of a dashboard as viewed on a System workstation monitor.

### DETAILED DESCRIPTION

As required, a detailed embodiment of the present invention is disclosed herein; however, it is to be understood that the disclosed embodiment is merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

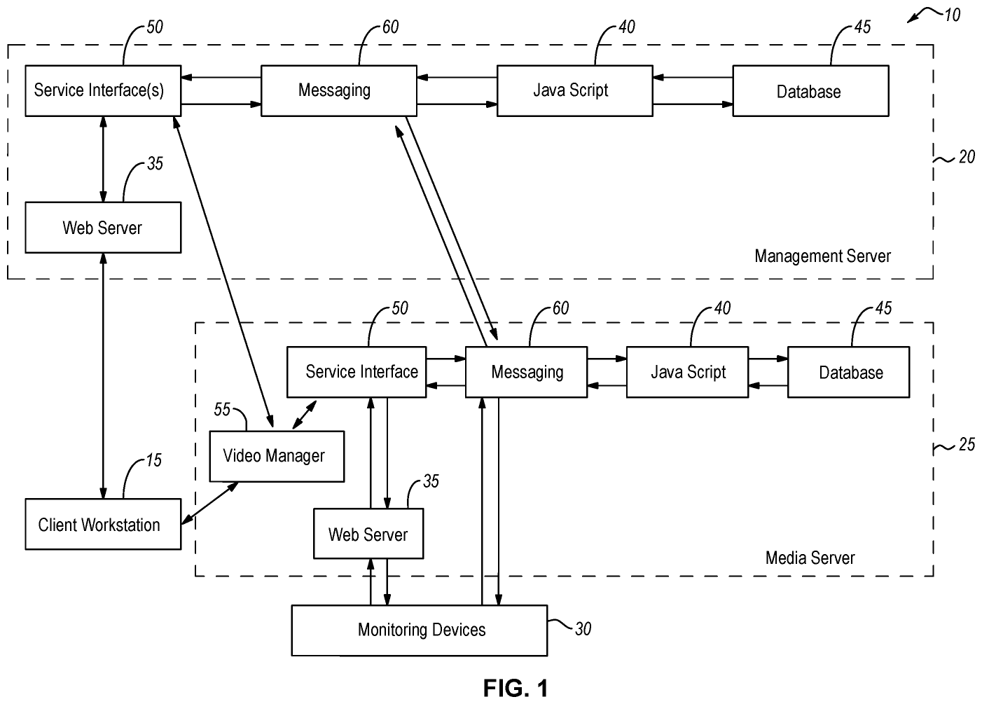

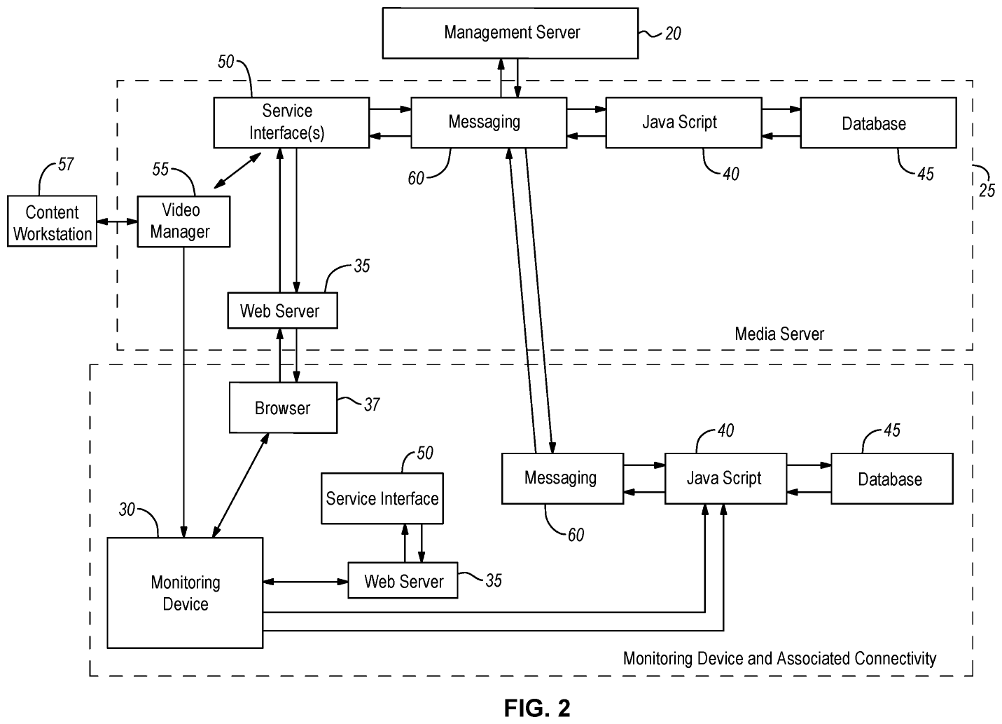

Embodiments of the present invention include a Remote Patient Monitoring System (System) 10, see FIGS. 1 and 2, for audiovisual observation and communication between a remote, centralized Patient Observation Station (Workstation) 15 and one or more patient hospital rooms in order to provide a means for continuous monitoring of each patient's condition and status, and to provide a means for patient and health care worker communication with the Patient Observation Station 15. Embodiments of the System 10 include a Management Server 20 to provide centralized operation and administration of one or more Media Servers 25. Each Media Server 25 provides centralized operation and administration of one or more Monitoring Devices 30, which may include platforms such as mobile wheeled carts, mobile wall units, and fixed cameras and intercoms.

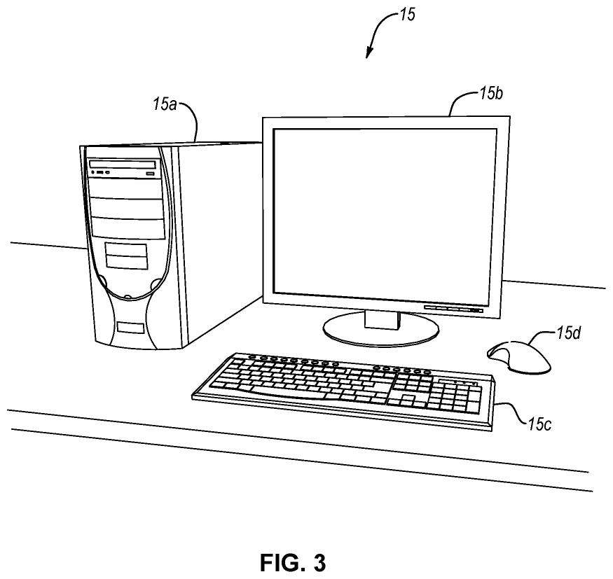

The Management Server 20 and Media Servers 25 are controlled and operated via a computer Workstation 15 that may be located at a Patient Observation Station, see FIG. 3. A Workstation 15 may comprise a personal computer 15a or other computing device including a screen or monitor 15b for observing patient room video, a CPU (not shown), interaction devices such as a keyboard 15c and mouse 15d, and a means for connecting electronically to the Management and Media Servers such as via Ethernet. Certain embodiments of a System may comprise more than one Management Server 20 for large scale implementations of the System 10.

Management Servers 20 and Media Servers 25 are typically physically installed within a user server room or data center, and preferably within a secured location, and will typically utilize a hospital or other facility's existing local wired and wireless network infrastructure to communicate with each other. A Management Server 20 may be deployed within the same Local Area Network (LAN) as the Media Servers 25 that it has been designated to manage to optimize secure and stable communication between the Management Server 20 and Media Servers 25 and to optimize System 10 performance. A Media Server 25 may be deployed within the same LAN as the Monitoring Devices 30 that it has been designated to manage to further optimize secure and stable communication between the Media Server 25 and associated Monitoring Devices 30 and to further optimize System 10 performance.

Some embodiments of a System 10 may comprise a centralized Management Server 20 assigned to Media Servers 25 located outside the LAN and in geographically separated locations. Multiple Management Servers 20 may function independently of other Management Servers 20 in a System 10 or may be controlled collectively via a Cloud Controller (not shown) deployed at a centralized data center or via a cloud service provider. In addition to providing for centralized operation and administration of Media Servers 25, a Management Server 30 is connected to and operated by one or more local or remote Workstations 15. An appropriate Management Server 20 may include a VMWare ESXi Linux Virtual Server.

Connectivity to Workstations 15 may be provided by a Management Server HTML5 compliant interface, such as a WebRTC HTML5 compliant interface. Embodiments of a Management Server 20 may be built upon a Linux-based platform designed for continuous operation. The Management Server 20 and Media Servers 25 may utilize technologies such as the NGINX High Performance Web Server platform 35, a cross-platform JavaScript run-time environment that executes JavaScript code server-side, such as NodeJS 40 utilizing an Express JS framework, a cross-platform document-oriented database program, such as MongoDB 45, and a service interface developed using a web application framework such as Angular 7 (or succeeding versions) 50.

Each Media Server 25 is associated with Monitoring Devices 30 and other media devices that it manages, but it must also be registered with and managed by a Management Server 20. In the smallest embodiments of a System 10, one Management Server 20 manages one Media Server 25. Certain embodiments of a Media Server 25 provide the primary HTML5 (or equivalent) based video and audio controls for the System 10, are built upon a Linux-based platform designed for continuous operation, and may be equipped with standards for HTML5 and WebRTC. Video and audio controls may be managed via a Video Manager 55 accessed via a Content Workstation 57 proximate or remote to the physical location of the Media Server 25.

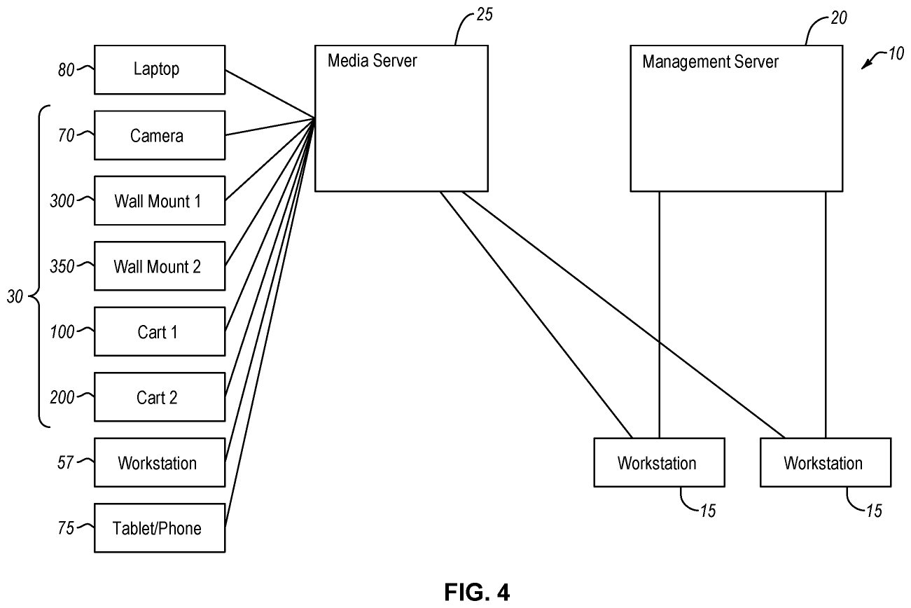

Media Servers 25 may be connected to various types of Monitoring Devices 30 described herein, such as stand-alone cameras 70, portable wall mounted assets 300 and 350, and PTZ cameras 170 and 225 and microphones 180 and 230 mounted on Mobile Carts 100 and 200, see FIG. 4. Additional media devices for audio and/video communication may be accessed by, or have access to, the Media Server 25, including Mobile Cart monitor cameras 132, tablets and smart phones 75 with cameras, and laptop/personal computers 80 equipped with cameras.

Some such media devices may communicate with the Media Server 25 via a TCP/IP connection or via Websockets 60. Object oriented techniques may be utilized within the Media Server 25 to maintain status and signaling for each media device to permit information to be tracked, logged, and to provide status and signaling between all components of the System 10. For some media device types, a Media Server 25 may present additional HTML5 WebRTC compliant interfaces to allow media devices to communicate with integrated System 10 applications. Media Servers 25 with full WebRTC support and client API's allow the System 10 to deliver advanced video applications and media processing capabilities, group communications, transcoding, recording, mixing, broadcasting, and routing of audiovisual flows.

Media Servers 25 may comprise a platform to provide high speed and efficient web services for delivery of HTML5 standards-based applications and services, such as a NGINX High Performance Web Server platform. The NGINX platform may be used to provide several different technologies for proxy, load balancing, and high availability as used by the System. Media Servers 25 may comprise a JavaScript runtime environment, such as NodeJS, and a web application server frame, such as ExpressJS, designed for building web applications based upon the JavaScript runtime environment. Embodiments of System Media Servers 25 may utilize both of these technologies for delivery of the Media Server web based application interface. Media Servers 25 may further comprise a cross-platform NoSQL database management system 45, such as MongoDB, that uses a document-oriented database model to support various forms of data. Web application framework technology 50, such as Angular 7 (or succeeding versions), is used by the System 10 to provide a robust web application implemented via the Media Servers 25.

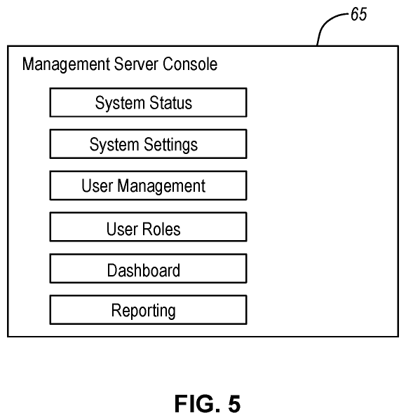

Certain embodiments of a Media Server 25 include an HTML based Media Server Console 65, FIG. 5, that can be used for administration, settings and managing the media assets that are connected and under control of the Media Server 25. The Media Server Console 65 may be accessed using standard networking connectivity from a laptop computer, desktop computer or other computing device via an Internet browser. When a Media Server 25 is first initialized, a settings page is provided to a System administrator/user allowing the required information to be specified to initialize the Media Server 25 platform. Subsequent changes to settings, such as changes to hostname, IP address, or other network settings, may also be entered via the Media Server Console 65.

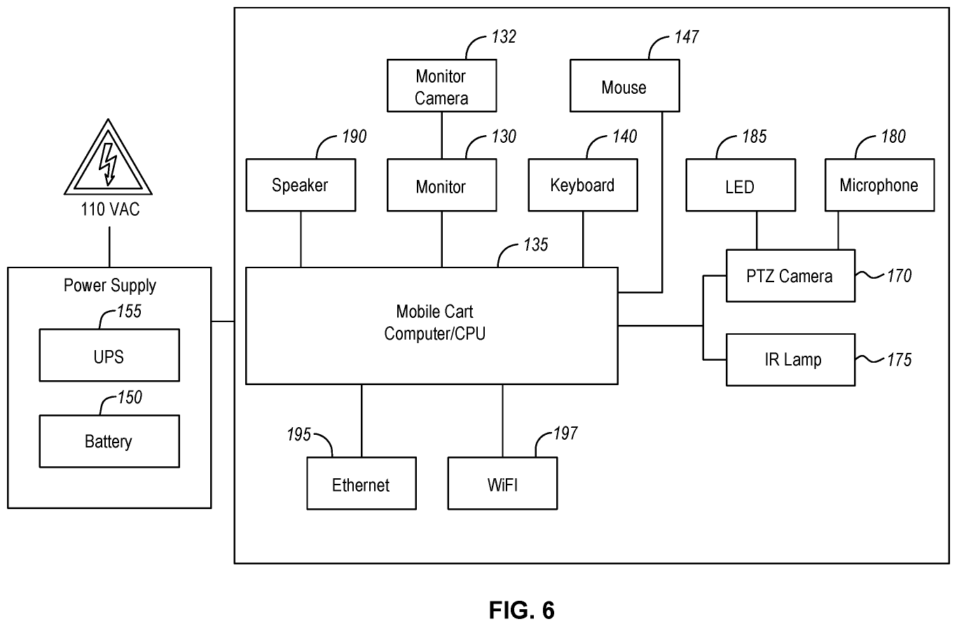

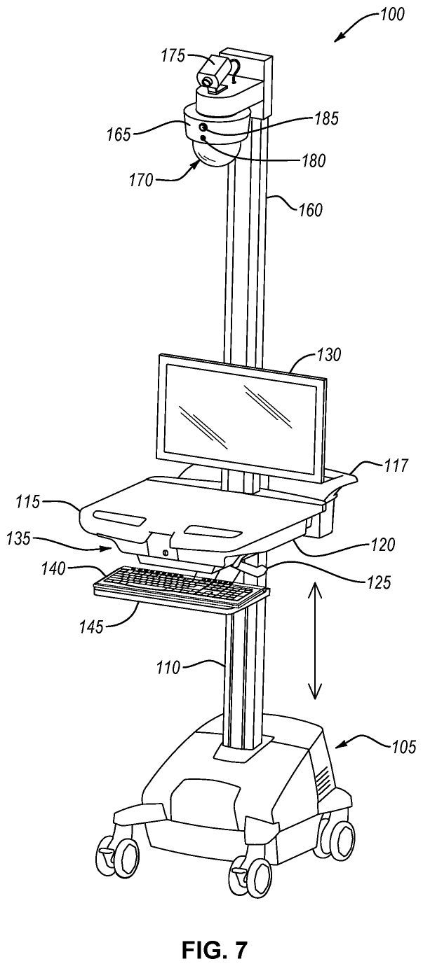

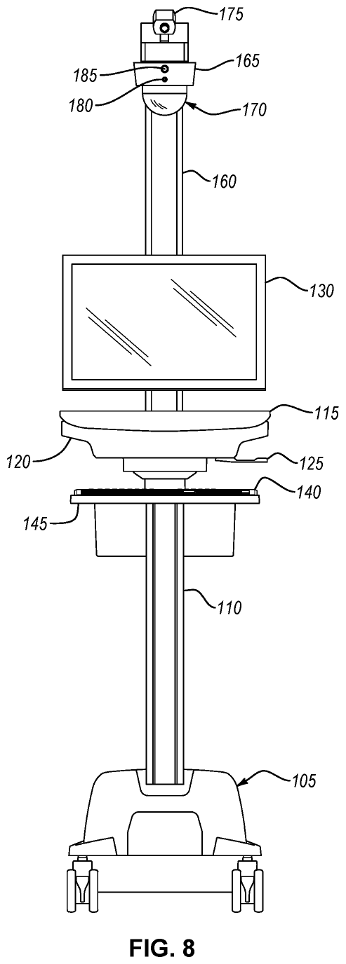

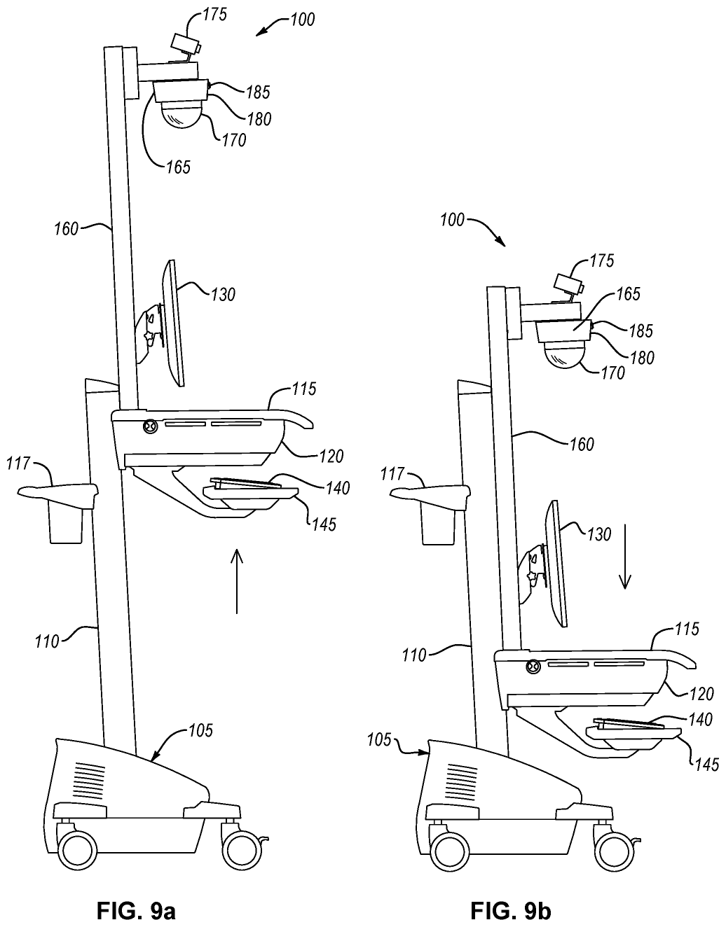

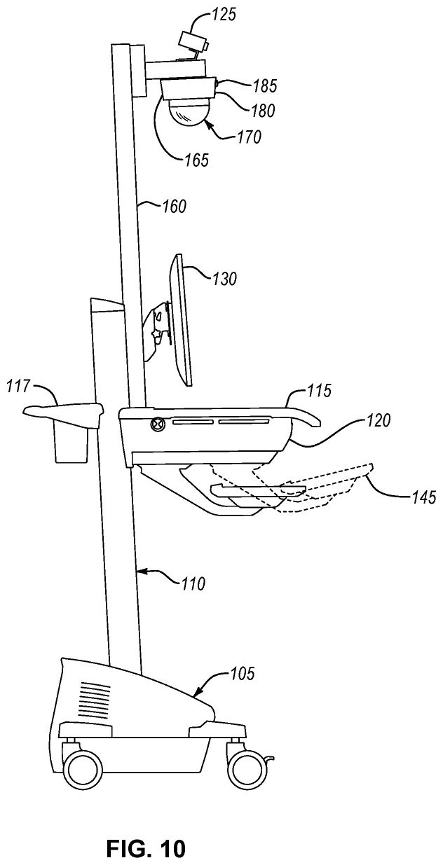

Monitoring Devices 30 such as a Mobile Cart are interfaced with the System 10 via the Media Server 25 and provide a mobile video and audio monitoring and communication system for use in locations remote from a Patient Observation Station 15, such as patient rooms. Certain embodiments of a Mobile Cart, such as a first Mobile Cart 100 (shown in FIGS. 7-10 and diagrammed in FIG. 6), include a wheeled base 105, which may house a battery 150 and an uninterruptible power supply (UPS) 155 for powering the electronic components of the first Mobile Cart 100. A tower 110 projects upward from the wheeled base, and a tray 115 projects generally horizontally outward and forward from the tower 110. A computer support housing 120 is attached to the underside of the tray 115. The tray 115 and computer support housing 120 assembly may be selectively lowered or raised along the tower 110, by selectively disengaging and engaging a locking lever 125. The first Mobile Cart 100 includes a computing device, such as a personal computer 135, mounted below the tray 115 within the computer support housing 120. A monitor 130 for providing a visual interface with the first Mobile Cart computer 135 is mounted on the tower 110 and facing forward and toward the tray 115 and the front of the first Mobile Cart 100. A monitor camera 132 (see FIG. 6) that may be used for video conferencing purposes is typically provided integrated with the monitor 130, as is known in the prior art. A keyboard 140 is mounted on a sliding keyboard tray 145 below the tray and computer housing. As indicated in FIG. 10, the keyboard tray 145 may be slid rearward underneath the tray during movement or storage of the first Mobile Cart 100 and slid forward for use. A computer mouse 147 (see FIG. 6) may also be provided. The keyboard 140 and mouse 147 may be connected to the first Mobile Cart 100 computer via USB cabling or other equivalent or practicable connections. In certain embodiments, a handle 117 for use in moving the first Mobile Cart 100 or other accessories may be mounted on the tower 110 or boom 160.

A substantially vertical boom 160 is attached to a rearward portion of the tray 115, and forward of the tower 110, so that the boom 160 can move upward or downward as the tray 115 is moved upward or downward along and relative to the tower 110. A pan-tilt-zoom (PTZ) camera 170 is mounted within a camera housing 165 near an upper end of the boom 160. The PTZ camera 170 captures video images of the area of a room forward and substantially to the sides of the first Mobile Cart 100 for transmission to an associated Media Server 25, Management Server 20 and Patient Observation Station 15. An IR lamp 175 is mounted at or near the top of the boom 160 for providing illumination to a room in the infrared (IR) spectrum. Although in preferred embodiments a PTZ camera 170 is used with the ability to capture images in low ambient light situations, the IR lamp 175 may be used to further illuminate a room without disturbing a sleeping patient. In these embodiments, a PTZ camera 170 is used with the ability to capture images in the IR spectrum. In certain embodiments of the invention, a non-PTZ fixed lens camera may be used.

The first Mobile Cart computer 135 includes a software platform, such as a Linux-based platform, to initialize the software components of the first Mobile Cart 100 and link it with it's designated Media Server 25 to present the first Mobile Cart's visual interface and application controls for normal operation. Following initial startup of the first Mobile Cart platform, the first Mobile Cart 100 will check its stored configuration and present a web based setup, such as an HTML5 web based setup, and setting display if further configuration is required. Using the keyboard 140 and mouse 147, the initial settings required may be entered to finalize setup and configuration and communication between the first Mobile Cart computer 135 and associated Media Server 25. Upon obtaining communication with the associated Media Server 25, the first Mobile Cart computer 135 will be registered with Media Server 25 and, using object-oriented techniques, the status of the first Mobile Cart computer 135 and associated devices, such as the PTZ camera 170, will be tracked and status will be updated in the associated Media Server 25 and Management Server 20.

In certain embodiments of a System 10, a patient observer manning a Patient Observation Station 15 can select via a Patient Observation Station dashboard, or other System graphic user interface, to darken their Workstation monitor 15b screen to allow the patient privacy by cutting or obscuring the video feed from the patient room to the Workstation. This is a timed event that is recorded and monitored by the System 10. This Privacy Mode is initiated by the patient observer and the System 10 will prompt the patient observer after a preselected period of time to restore the video feed from the room to their Monitor 15b screen. When Privacy Mode has been initiated a confirmatory signal is provided in the patient room via a privacy mode indicator LED 185 that is illuminated when the video feed from the PTZ camera 170 or 225 is displayed on the Monitor 15b and is turned off and not illuminated when Privacy Mode has been initiated, thus confirming to the patient and others in the patient room that no video images from the room are being viewed by others.

Audio monitoring of the patient room may be provided by a microphone 180 that, along with the privacy mode indicator LED 185, may be mounted or built into the camera housing 165.

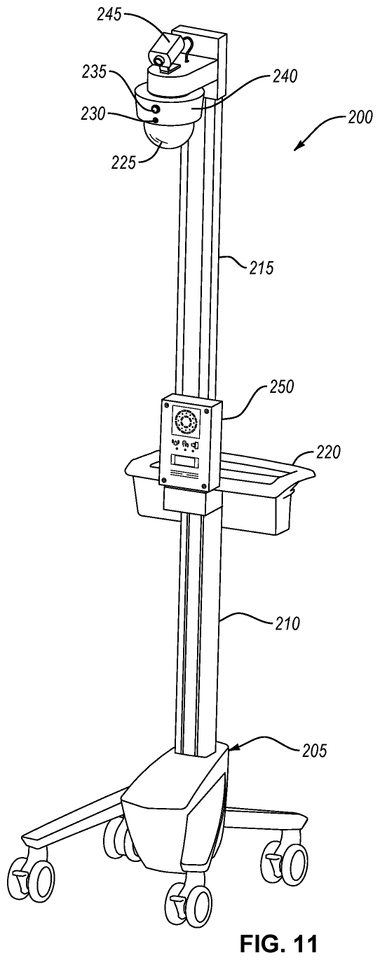

Certain embodiments of a System 10 may include and employ an alternative embodiment of a Mobile Cart such as the second Mobile Cart 200 illustrated in FIG. 11. A second Mobile Cart 200, as described herein, may include a wheeled base 205, tower 210, tower extension or boom 215, handle 220, PTZ Camera 225, and microphone 230 and privacy mode indicator LED 235 built into the camera housing 240. Certain embodiments of the second Mobile Cart 200 may also include an IR lamp 245. Typically, an intercom 250 is provided for selected communications with an operator manning the Patient Observation Station 15.

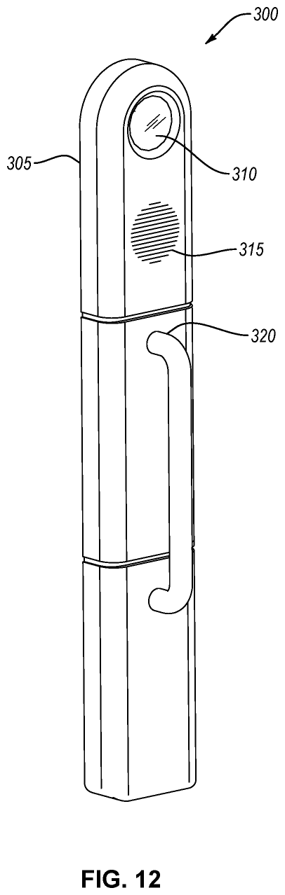

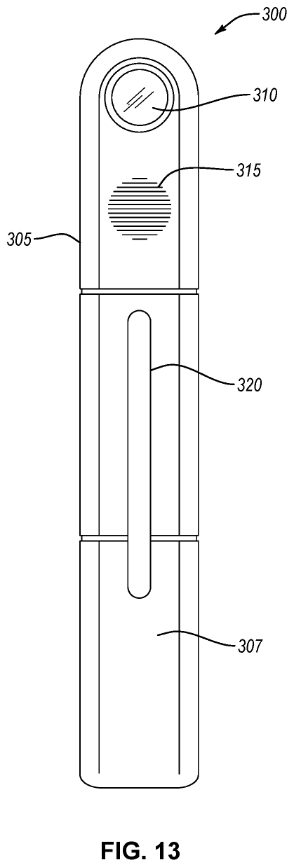

Among type of Monitoring Device 30 that may be provided by the System 10 includes a Portable Wall Asset or Unit 300, see FIGS. 12-14b. In certain embodiments, a Portable Wall Unit 300 comprises an elongated, generally rectangular, wall unit housing 305. The housing 305 will typically comprise plastic or other non-porous, resilient material that is resistant to damage and readily cleaned and disinfected. The Portable Wall Unit 300 comprises a camera 310 that is connected via WIFI or other practicable means to Media Server 25 for providing a video fee from a patient room. A camera 310 may comprise a 4K resolution camera at full frame rate, with or without pan-tilt-zoom. In some embodiments of the Portable Wall Unit 300, the camera 310 includes the ability to obtain video imagery in low ambient light. In some embodiments, the camera 310 is able to obtain video imagery via IR illumination. Integrated hardware in the Portable Wall Unit 300 may include a privacy mode LED, microphone, speaker 315, power supply and CPU. A handle 320 mounted to the front surface 307 of the wall unit housing 305 provides a means for grasping and carrying the Portable Wall Unit 300 from one location to another.

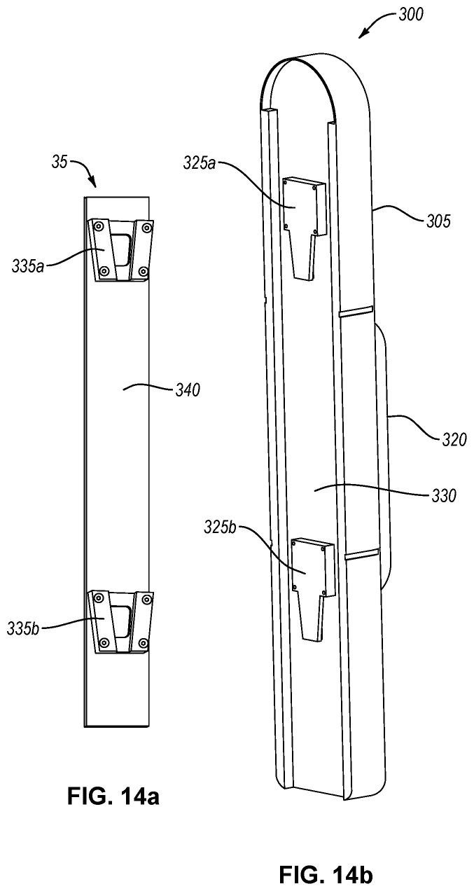

The Portable Wall Unit 300 may be removably mounted to a wall via a bracket system. As shown in FIGS. 14a and 14b, a pair of male brackets 325a and 325b are attached spaced apart and one above the other to the back side 330 of the Portable Wall Unit 300. A pair of cooperating female brackets 335a and 335b are attached spaced apart and one above the other to the front face 340 of a rectangular wall mount 345. The wall mount 345 may be mounted at a desired position on the wall of a hospital room or other surface using screws or other fasteners or fastening means sufficient to allow the wall mount 345 and brackets to reliably bear the weight of the Portable Wall Unit 300. The female brackets 335a and 335b are spaced apart the same distance as the cooperating male brackets 325a and 325b so that when the Portable Wall Unit 300 is lifted and positioned so that the male brackets are aligned above the female brackets, each male bracket will slide into and engage its cooperating female bracket.

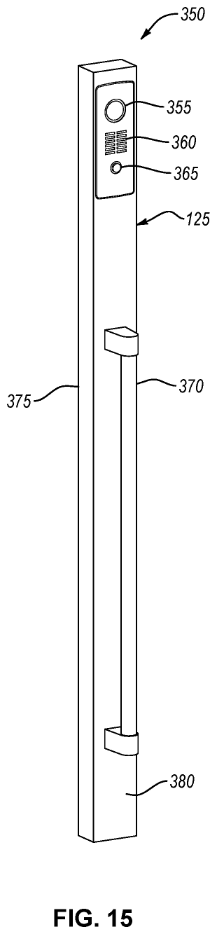

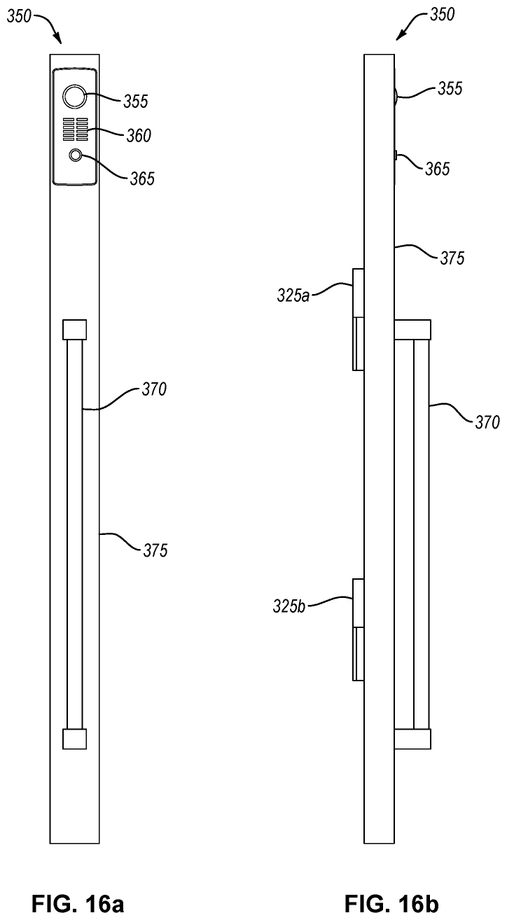

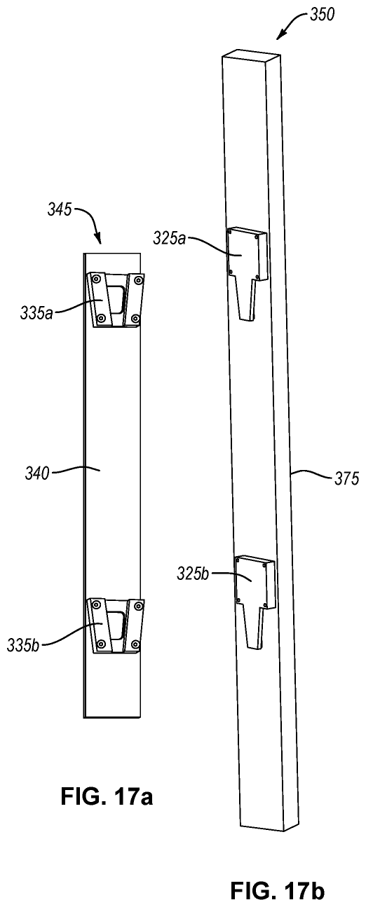

An alternative embodiment of a Portable Wall Unit 350 is illustrated in FIGS. 15-17b. This Portable Wall Unit 300 includes a video camera 355, speaker 360 and privacy mode indicator LED 365. An elongated handle 370 is attached to the front face 380 of the generally rectangular housing 375. As shown, this Portable Wall Unit 350 includes the same bracket system as Portable Wall Unit 300.

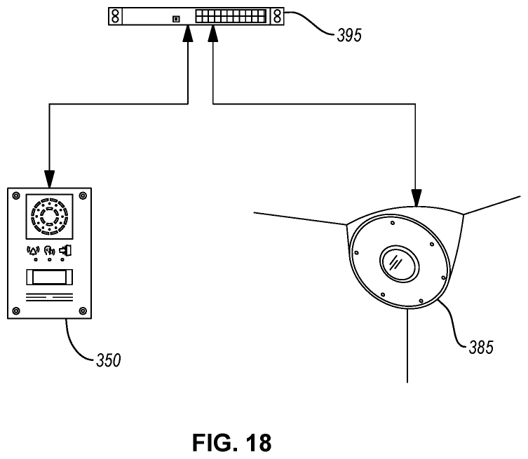

Some embodiments of a System 10 may include alternative additional Monitoring Devices 30 such as a stand-alone, fixed-position PTZ camera mounted on a ceiling or wall of a patient room, or other room to be monitored, similar to the PTZ cameras 170 and 225 mounted on Mobile Carts 100 and 200. Some embodiments of a System 10 may include a stand-alone, fixed-position wide angle camera 385, and a stand-alone intercom 390 that communicate with the Media Server 25 via an Ethernet Switch 395, FIG. 18.

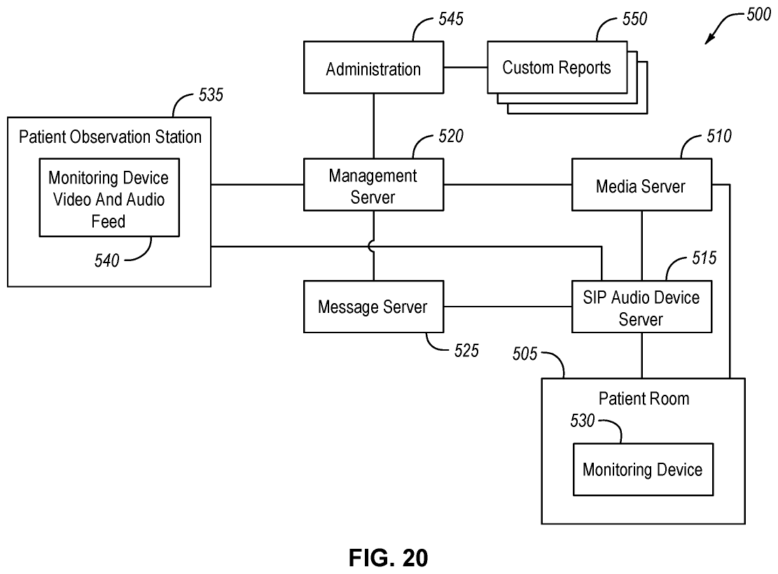

FIG. 20 is a diagram illustrating components of an alternative embodiment of a System 500 according to the present invention. This embodiment of the System 500 provides a means for communicating via audio and receiving video from a patient's room 505. A Media Server 510, such as a Cisco VSM server, comprises a platform used to manage the video devices installed in the patient rooms. A SIP Audio Device Server 515, such as a Cisco CUCM server, comprises a platform used to manage the SIP audio devices installed in the patient rooms 505. A Management Server 520 interfaces with the Media Server 510 and SIP Audio Device Server 515 to manage/integrate video and privacy controls and audio controls. A Message Server 525, such as a Singlewire server, interfaces with the SIP Audio Device Server 515 and Management Server 520 to provide multilingual prerecorded messages and audible alerts to the Monitoring Device 530 (e.g. Mobile Cart asset, Portable Wall-mounted asset or Fixed Camera asset) in the patient room. A Patient Observation Station 535 interfaces with the Management Server 520, Media Server 510 and SIP Audio Device Server 515 to receive Monitoring Device 530 video and audio feed 540 from the patient room 505 and provides a customized graphic user interface (GUI) or dashboard with which an operator monitors patients remotely. Via the Patient Observation Station 535, an Observation Technician can enroll new patients in the System 500. Typically, the system will prompt Observation Technicians to perform patient surveys every 15 minutes. An Observation Technician can contact the patient room 10 via audio by pushing a button to engage audio transmission from the station 60 to the patient room 10, or via other means. In certain embodiments, the Observation Technician can initiate privacy mode for the patient room by cutting the video feed remotely. In such instances, a reminder message will be displayed at the station 60 to re-engage video feed if privacy has been enabled for an extended duration. Via the station GUI, an Observation Technician can perform VPO Asset Transfers to easily move the patient out of a room or to another room. The Observation Technician can view key patient information and update the nurse and runner information. System administrators may use a dedicated Workstation or non-dedicated PC or laptop to access the Management Server 520 via the LAN to perform Administration 545 tasks such as run Custom Reports 550.

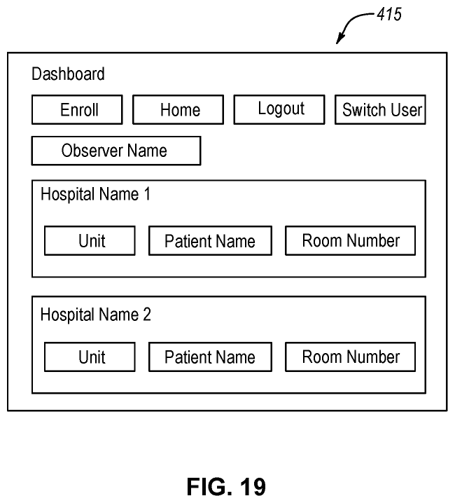

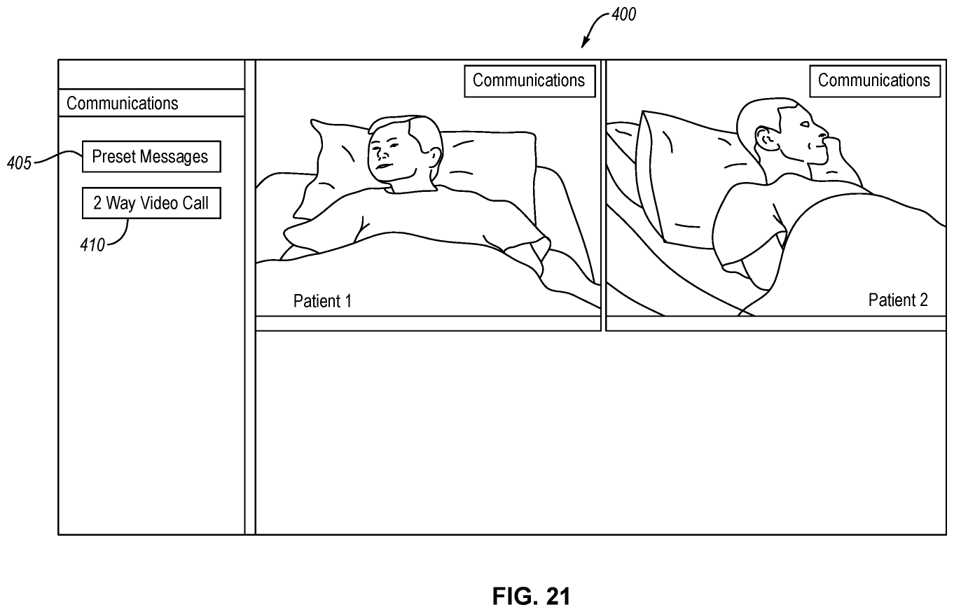

FIG. 21 is a diagram illustrating a communication control screen 400 of a dashboard as viewed on a System workstation monitor 15b or other System interface. An operator may select a patient room and initiate communications with a patient or other person in the room, or respond, by either selecting to send a Preset Message 405 or to initiate a 2-way video call 410. FIG. 19 is a diagram of a System dashboard 415 that provides selections for an operator/patient observer to change the responsibility for viewing a set of patients from one patient observer to another.

It is to be understood that while certain forms of this invention have been illustrated and described, it is not limited thereto except insofar as such limitations are included in the following claims and allowable equivalents thereof.

Having thus described the invention, what is claimed as new and desired to be secured by Letters Patent is:

1. A patient monitoring system comprising:
a management server,
a media server,
a monitoring device, said monitoring device including a computer processor, a video camera, a microphone, and wireless router for transmitting video, audio and data signals to said media server and management server.

2. The patient monitoring system of claim 1 wherein video signals from said video camera are conveyed via said media server to a patient observation station for viewing by a patient observer.

3. The patient monitoring system of claim 1 wherein audio signals from said microphone are conveyed via said media server to a patient observation station for listening by a patient observer.

4. The patient monitoring system of claim 2 wherein a patient observer may select a privacy mode to selectively darken a portion of a patient observation station monitor screen in order to obscure video images from a patient room.

5. The patient monitoring system of claim 4 wherein selecting a privacy mode causes a privacy indicator light to dim in a patient room, thereby confirming to the room occupants that video signals taken by said camera are no longer being displayed upon said monitor screen.
